(19) United States
(12) Patent Application Publication
(10) Pub. No.: US 2006/0073175 A1
(43) Pub. Date: Apr. 6, 2006

(54) Methods and formulations for delivery of pharmacologically active agents

(75) Inventors: Patrick Soon-Shiong, Los Angeles, CA (US); Neil P. Desai, Los Angeles, CA (US)

Correspondence Address: FOLEY & LARDNER LLP, P.O. BOX 80278, SAN DIEGO, CA, 92138-0278, US

(73) Assignee: American Bioscience, Inc.

(21) Appl. No.: 11/240,940

(22) Filed: Sep. 29, 2005

Related U.S. Application Data
(63) Continuation of application No. 10/146,706, filed on May 14, 2002, now abandoned.
Application No. 10/146,706 is a continuation-in-part of application No. 09/628,388, filed on Aug. 1, 2000, now Pat. No. 6,506,405.
Application No. 09/628,388 is a division of application No. 08/926,155, filed on Sep. 9, 1997, now Pat. No. 6,096,331.
Application No. 08/926,155 is a continuation-in-part of application No. 08/720,756, filed on Oct. 1, 1996, now Pat. No. 5,916,596.
Application No. 08/720,756 is a continuation-in-part of application No. 08/485,448, filed on Jun. 7, 1995, now Pat. No. 5,665,382.
Application No. 08/485,448 is a continuation-in-part of application No. 08/200,235, filed on Feb. 22, 1994, now Pat. No. 5,498,421.
Application No. 08/200,235 is a continuation-in-part of application No. 08/023,698, filed on Feb. 22, 1993, now Pat. No. 5,439,686.

(51) Int. Cl. A61K 9/00

(52) U.S. Cl. 424/400

(57) ABSTRACT

In accordance with the present invention, novel formulations have been developed which are much more effective for the delivery of hydrophobic drugs to patients in need thereof than are prior art formulations. Invention formulations are capable of delivering more drug in shorter periods of time, with reduced side effects caused by the pharmaceutical carrier employed for delivery.

### RELATED APPLICATIONS

The present application is a continuation-in-part of U.S. Ser. No. 09/628,388, filed Aug. 1, 2000, now pending, which is a divisional of U.S. Ser. No 08/926,155, now issued as U.S. Pat. No. 6,096,331, which is a continuation-in-part of U.S. Ser. No. 08/720,756, filed Oct. 1, 1996, now issued as U.S. Pat. No. 5,916,596, and U.S. Ser. No. 08/485,448, filed Jun. 7, 1995, now U.S. Pat. No. 5,665,382, which is, in turn, a continuation-in-part of U.S. Ser. No. 08/200,235, filed Feb. 22, 1994, now issued as U.S. Pat. No. 5,498,421, which is, in turn, a continuation-in-part of U.S. Ser. No. 08/023,698, filed Feb. 22, 1993, now issued as U.S. Pat. No. 5,439,626 and U.S. Ser. No. 08/035,150, filed Mar. 26, 1993, now issued as U.S. Pat. No. 5,362,478, the contents of each of which are hereby incorporated by reference herein in their entirety.

### FIELD OF THE INVENTION

The present invention relates to novel formulations of pharmacologically active agents and methods for the delivery of such agents to subjects in need thereof.

### BACKGROUND OF THE INVENTION

In the quest for next generation therapies to treat cancer, scientists often discover promising compounds only to find that the molecule is highly insoluble in water, and hence impossible to deliver intravenously. Such was the problem with paclitaxel, an extremely effective anti-tumor agent discovered over a quarter century ago by the National Cancer Institute. Despite almost 30 years of effort, the only method currently approved to address this problem of water-insolubility of paclitaxel is the use of a toxic solvent (cremophor) to dissolve the drug, and administer this solvent-paclitaxel mixture over many hours using specialized intra-venous tubing sets to prevent the leaching of plasticizers. This solvent-drug mixture, currently marketed in branded and generic forms, has become the most widely used anti-cancer agent as it has shown activity in breast, lung and ovarian cancer and is undergoing multiple clinical trials exploring its application in combination with other drugs for other solid tumors.

The cremophor formulation of paclitaxel is associated with significant side-effects including life-threatening allergic reactions requiring the need for steroid pre-treatment for every patient receiving the drug, and severe infections as a result of lowering of white blood cells requiring the need for expensive blood cell growth factors. Ultimately these toxicities result in dose-limitation of cremophor-based paclitaxel formulations, thus limiting the full potential of the very effective paclitaxel molecule.

While the above toxic side effects of cremophor paclitaxel formulations are well known, it has not been widely recognized by scientists in the field that the presence of cremophor creates a more serious impediment to realizing the maximal potential of paclitaxel by entrapping paclitaxel within the hydrophobic cores of cremophor micelles within microdroplets in the blood-stream. The entrapment effect of cremophor is dependent on cremophor concentration. Thus, increasing the doses of cremophor solutions of paclitaxel can-potentially worsen the entrapment by raising the concentration of cremophor, leading to higher toxcities but none of the potential benefits of higher doses of paclitaxel, since much of the active molecule is unavailable to the intra-cellular space, where it is needed to act.

This entrapment of paclitaxel by cremophor has a profound effect on the intra-cellular availability of the active molecule and hence may have significant clinical implications in terms of clinical outcome. Accordingly, there is a need in the art for new formulations for the delivery of substantially water insoluble pharmacologically active agents, such as paclitaxel, which do not suffer from the drawbacks of cremophor.

### BRIEF DESCRIPTION OF THE INVENTION

In accordance with the present invention, novel formulations have been developed which are much more effective for the delivery of hydrophobic drugs to patients in need thereof than are prior art formulations. Invention formulations are capable of delivering more drug in shorter periods of time, with reduced side effects caused by the pharmaceutical carrier employed for delivery.

### BRIEF DESCRIPTION OF THE FIGURES

FIG. 1 collectively compares the plasma kinetics of radiolabelled paclitaxel when administered to a mouse model as part of a Taxol formulation (closed squares) or as part of in invention formulation (diamonds; ABI-007). FIG. 1A indicates plasma radioactivity measured up to 0.5 hours after administration. FIG. 1B indicates plasma radioactivity measured up to 24 hours after administration. Inspection of the figure reveals that 2-5 fold higher levels of paclitaxel are retained in the plasma up to 3 hours after administration when paclitaxel is administered in a cremophor-based formulation (Taxol). Due to the reduced rate of metabolism for ABI-007, plasma levels of paclitaxel are higher after 8 hours when administered in an invention formulation, relative to a cremophor-based formulation.

FIG. 2 compares the partitioning of paclitaxel between red blood cells and plasma when administered to a mouse model as part of a Taxol formulation (closed squares) or as part of in invention formulation (diamonds; ABI-007). Inspection of the figure reveals that the blood/plasma ratio for paclitaxel administered as part of a cremophor-based formulation (Taxol) in the first 3 hours after administration is about 1.5-2, indicating that the majority of paclitaxel is retained in the plasma due to micellar formation with cremophor. In addition, it is seen that paclitaxel in a cremophor-based formulation does not significantly partition into the red blood cells. In contrast, paclitaxel administered as part of an invention formulation readily partitions into the red blood cells.

FIG. 3 summarizes tumor/plasma partitioning kinetics of paclitaxel when administered to a mouse model as part of a Taxol formulation (closed squares) or as part of in invention formulation (diamonds; ABI-007). It is seen that the tumor/plasma ratio of paclitaxel increases significantly over the first 3 hours when as part of an invention formulation, as opposed to a Taxol formulation.

FIG. 4 compares the response of mammary carcinoma in a mouse model to exposure to ABI-007 or Taxol.

FIG. 5 compares the response of ovarian carcinoma in a mouse model to exposure to ABI-007 or Taxol.

FIG. 6 compares the response of prostate tumors in a mouse model to exposure to ABI-007 or Taxol.

FIG. 7 compares the response of colon tumors in a mouse model to exposure to ABI-007 or Taxol.

FIG. 8 compares the response of lung tumors in a mouse model to exposure to ABI-007 or Taxol.

### DETAILED DESCRIPTION OF THE INVENTION

In accordance with the present invention, there are provided methods for the delivery of a substantially water insoluble pharmacologically active agent to a subject in need thereof, said method comprising combining said agent with an effective amount of a pharmaceutically acceptable carrier which is substantially free of micelle-forming components, and administering an effective amount of said combination to said subject.

As readily recognized by those of skill in the art, a wide variety of pharmacologically active agents are contemplated for use in the practice of the present invention. A presently preferred agent contemplated for use herein is paclitaxel.

Pharmaceutically acceptable carriers contemplated for use in the practice of the present invention are biocompatible materials such as albumin.

Micelle-forming components which are preferably avoided in the practice of the present invention are surface active materials which are commonly used to assist in solubilizing substantially insoluble compounds in aqueous media, such as, for example, cremophor.

Invention combination of active agent and pharmaceutically acceptable carrier can be administered in a variety of ways, such as, for example, by oral, intravenous, subcutaneous, intraperitoneal, intrathecal, intramuscular, intracranial, inhalational, topical, transdermal, rectal, or pessary routes of administration, and the like.

In accordance with another embodiment of the present invention, there are provided methods to reduce entrapment of a substantially water insoluble pharmacologically active agent in vehicle employed for delivery thereof, said method comprising combining said agent with a pharmaceutically acceptable carrier which is substantially free of micelle-forming components prior to delivery thereof.

Presently preferred pharmaceutically acceptable carriers contemplated for use herein are those having substantially lower affinity for said agent than does the micelle-forming component. Thus, for example, while cremophor has the benefit of aiding in the solubilization of agent, it has the disadvantage of having a substantial affinity for the agent, so that release of the agent from the carrier becomes a limitation on the bioavailability of the agent. In contrast, carriers contemplated herein, such as, for example, albumin, readily release the active agent to the active site and are thus much more effective for treatment of a variety of conditions.

In accordance with yet another embodiment of the present invention, there are provided methods to reduce entrapment of a substantially water insoluble pharmacologically active agent in vehicle employed for delivery thereof, said method comprising employing pharmaceutically acceptable carriers which are substantially free of micelle-forming components in aqueous media as the vehicle for delivery of said agent.

In accordance with still another embodiment of the present invention, there are provided methods to prolong exposure of a subject to a substantially water insoluble pharmacologically active agent upon administration thereof to a subject in need thereof, said method comprising combining said agent with pharmaceutically acceptable carrier(s) which is(are) substantially free of micelle-forming components prior to delivery thereof.

In accordance with a further embodiment of the present invention, there are provided methods to facilitate transport of a substantially-water insoluble pharmacologically active agent across cell membranes upon administration thereof to a subject in need thereof, said method comprising combining said agent with pharmaceutically acceptable carrier(s) which is(are) substantially free of micelle-forming components prior to delivery thereof.

In accordance with a still further embodiment of the present invention, there are provided methods to facilitate transport of a substantially water insoluble pharmacologically active agent into the cellular compartment upon administration thereof to a subject in need thereof, said method comprising combining said agent with pharmaceutically acceptable carrier(s) which is(are) substantially free of micelle-forming components prior to delivery thereof.

In accordance with another embodiment of the present invention, there are provided formulations comprising a substantially water insoluble pharmacologically active agent and a pharmaceutically acceptable carrier which is substantially free of micelle-forming components, wherein said formulation provides a higher concentration of said agent in the cellular compartment than a formulation of the same agent with a micelle-forming component.

In accordance with yet another embodiment of the present invention, there are provided formulations comprising a substantially water insoluble pharmacologically active agent and a pharmaceutically acceptable carrier which is substantially free of micelle-forming components, wherein said formulation provides increased intra-cellular availability of said agent relative to a formulation of the same agent with a micelle-forming component.

In accordance with still another embodiment of the present invention, there are provided formulations comprising a substantially water insoluble pharmacologically active agent and a pharmaceutically acceptable carrier which is substantially free of micelle-forming components, wherein said formulation provides prolonged activity of said agent relative to a formulation of the same agent with a micelle-forming component.

In accordance with a further embodiment of the present invention, there are provided formulations comprising a substantially water insoluble pharmacologically active agent and a pharmaceutically acceptable carrier which- is substantially free of micelle-forming components, wherein said formulation facilitates delivery of said agent to red blood cells.

In accordance with another embodiment of the present invention, there are provided formulations comprising a substantially water insoluble pharmacologically active agent and a pharmaceutically acceptable carrier which is substantially free of micelle-forming components, wherein said formulation releases a portion of said agent contained therein to the lipid membrane of a cell.

In accordance with yet another embodiment of the present invention, there are provided formulations comprising a substantially water insoluble pharmacologically active agent and a pharmaceutically acceptable carrier which is substantially free of micelle-forming components, wherein said formulation provides reduced levels of said agent in the bloodstream relative to a formulation of the same agent with a micelle-forming component.

In accordance with still another embodiment of the present invention, there are provided formulations comprising a substantially water insoluble pharmacologically active agent and a pharmaceutically acceptable carrier which is substantially free of micelle-forming components, wherein said formulation delivers said agent to the bloodstream over an extended period of time relative to a formulation of the same agent with a micelle-forming component.

In accordance with a further embodiment of the present invention, there are provided formulations comprising a substantially water insoluble pharmacologically active agent and a pharmaceutically acceptable carrier which is substantially free of micelle-forming components, wherein the rate of metabolism of said agent in said formulation is reduced relative to the rate of metabolism of said agent in a formulation with a micelle-forming component.

In accordance with another embodiment of the present invention, there are provided formulations comprising a substantially water insoluble pharmacologically active agent and a pharmaceutically acceptable carrier which is substantially free of micelle-formiing components, wherein said agent has a longer half life in said formulation relative to the half life of said agent in a formulation with a micelle-forming component.

In accordance with yet another embodiment of the present invention, there are provided formulations comprising a substantially water insoluble pharmacologically active agent and a pharmaceutically acceptable carrier which is substantially free of micelle-forming components, wherein said formulation provides a higher red blood cell/plasma ratio of said agent than does a formulation of the same agent with a micelle-forming component.

In accordance with still another embodiment of the present invention, there are provided formulations comprising a substantially water insoluble pharmacologically active agent and a pharmaceutically acceptable carrier which is substantially free of micelle-forming components, wherein said formulation provides a higher tumor/plasma ratio of said agent than does a formulation of the same agent with a micelle-forming component.

In accordance with a further embodiment of the present invention, there are provided formulations comprising a substantially water insoluble pharmacologically active agent and a pharmaceutically acceptable carrier which is substantially free of micelle-forming components, wherein the area under the curve for delivery of said agent to a tumor via said formulation is higher than the area under the curve for delivery of said agent to a tumor via a formulation of the same agent with a micelle-forming component.

In accordance with a still further embodiment of the present invention, there are provided formulations comprising a substantially water insoluble pharmacologically active agent and a pharmaceutically acceptable carrier which is substantially free of micelle-forming components, wherein said formulation provides a higher concentration maximum (Cₘₐₓ) for said agent in tumor cells than does a formulation of the same agent with a micelle-forming component.

In accordance with another embodiment of the present invention, there are provided formulations comprising a substantially water insoluble pharmacologically active agent and a pharmaceutically acceptable carrier which is substantially free of micelle-forming components, wherein said formulation provides a lower concentration maximum (Cₘₐₓ) for said agent in plasma than does a formulation of the same agent with a micelle-forming component.

In accordance with still another embodiment of the present invention, there are provided formulations comprising a substantially water insoluble pharmacologically active agent and a pharmaceutically acceptable carrier which is substantially free of micelle-forming components, wherein said formulation provides more rapid uptake of said agent by tumor cells than does a formulation of the same agent with a micelle-forming component.

In accordance with yet another embodiment of the present invention, there are provided formulations comprising a substantially water insoluble pharmacologically active agent and a pharmaceutically acceptable carrier which is substantially free of micelle-forming components, wherein said formulation enhances delivery of said agent to tissue, relative to a formulation of the same agent with a micelle-forming component.

Tissues contemplated for treatment according to the invention include tumors, peritoneal tissue, bladder tissue, lung tissue, and the like.

ABI-007 is a proprietary, cremophor-free, albumin-based paclitaxel nanoparticle, 1/100^{th} the size of a single red blood cell. Based on several Phase I studies, it has been shown that ABI-007 can be administered rapidly without the need for steroid pre-treatment and without the need for G-CSF at a maximum tolerated dose of 300 mg/m given every 3 weeks. This is a significantly higher dose than is approved for cremophor-based paclitaxel formulations (Taxol) of 175 mg/m² .

In accordance with the present invention, it has been discovered that ABI-007 acts as a novel biologic nano-transporter for hydrophobic drugs such as paclitaxel, with the capabilities of rapidly releasing paclitaxel to the cellular compartment and increasing intra-cellular availability of the active drug, where it is needed in order to have its chemo-therapeutic effect. Furthermore, through the use of the red blood cell as a secondary storage vehicle it has been discovered that in addition to the rapid and increased availability of paclitaxel at the intra-cellullar level, by the recruitment of circulating red blood cells, ABI-007 further provides a significant prolonged activity of the parent molecule with sustained in-vivo release. These novel mechanisms for rapid and increased intra-cellular availability of the drug at the tumor site, together with sustained trafficking of the non-metabolized paclitaxel, has potentially significant implications for the clinical outcome in the treatment of solid tumors. Indeed. the pre-clinical and Phase II clinical data presented below supports this notion.

By taking advantage of the differences in binding affinities of albumin and the lipid bi-layer of cell membranes for hydrophobic paclitaxel, the drug-bearing albumin nanoparticle (ABI-007) would rapidly release a portion of its hydrophobic paclitaxel cargo to the lipid membrane of a cell.

In the vascular compartment, the first cell encountered is the red blood cell. In accordance with the present invention, the red blood cell has been found to rapidly compartmentalize the paclitaxel molecule. Since the red blood cell has no nucleus and hence no microtubulin to which the paclitaxel molecule can bind, nor any degradation machinery within its core, this cell serves as an ideal secondary storage vehicle for the active paclitaxel, accounting in part for the prolonged activity of paclitaxel noted with ABI-007.

Following partitioning of a portion of its paclitaxel payload to the circulating red blood cells, the nanoparticle is carried by the blood-stream to the hypervasular tumor, where paclitaxel is rapidly transferred to the tumor cell-membrane, again due to the differences in binding affinity. It has been well established by other groups that the hydrostatic pressure within these tumor cells is abnormally higher than the surrounding interstitium and vascular space. This abnormally high pressure, together with the fact that the vessels associated with tumors are also abnormally leaky, creates a barrier to the delivery of chemotherapeutic agents to the tumor cell. Thus, under these circumstances it is imperative that the hydrophobic paclitaxel be released rapidly to the lipid cell membrane and be bound by the microtubules within the nuclues before the drug is ejected from the tumor. Evidence presented herein indicates that ABI-007 provides that opportunity by the ability to rapidly release the hydrophobic molecule. In contrast, cremophor-based formulations entrap the paclitaxel, limiting the ability of the drug to partition into cells. This difference may have important clinical implications and may account in part for the positive data noted in the Phase II studies of ABI-007 in metastatic breast cancer and the evidence for responses in patients who had previously failed Taxol therapy

As the nanoparticle depeletes itself of paclitaxel, into the cellular compartment within the first 3-8 hours following infusion, the plasma concentartion of paclitaxel diminshes. At this juncture, paclitaxel (still in its active, non-metabolized form) follows the concentration gradient and is now transferred to albumin again, and is again carried to the tumor bed. Thus, a prolonged half-life of paclitaxel has been achieved, with sustained release and ultimately higher tumor concentration of the drug.

The invention will now be described in greater detail by reference to the following non-limiting examples.

### EXAMPLE 1

### Preclinical Studies Confirm the Modulation of Paclitaxel Release by the Protein Nanosphere and Increased Efficacy of Equi-Dose of ABI-007 vs Taxol

Using radio labeled paclitaxel, the enahanced intra-cellular availability of paclitaxel has been confirmed following injection of ABI-007. In addition, the entrapment of Cremophor-bound paclitaxel has also been confirmed. This difference in findings correlates with in-vivo studies in mice bearing human breast cancer, with the finding that ABI-007 at equi-dose to Taxol, resulted in improved outcomes, that these 130 nanometer size particles distributed throughout the body.

Thus, human MX-1 mammary tumor fragments were implanted subcutaneously in female athymic mice. Radiolabelled drug was administered when tumors reached about 500 mm³. Tritium-labelled ABI-007 or tritium-labelled Taxol were administered at a dose of 20 mg/kg. Both groups received about 7-10 μCi/mouse of tritium-labelled paclitaxel. Saline was used as the diluent for both drugs. At various time points (5 min, 15 min, 30 min, 1 hr, 3 hr, 8 hr and 24 hr), 4 animals were sacrificed, then blood samples and tumor were recovered for radioactivity assessment.

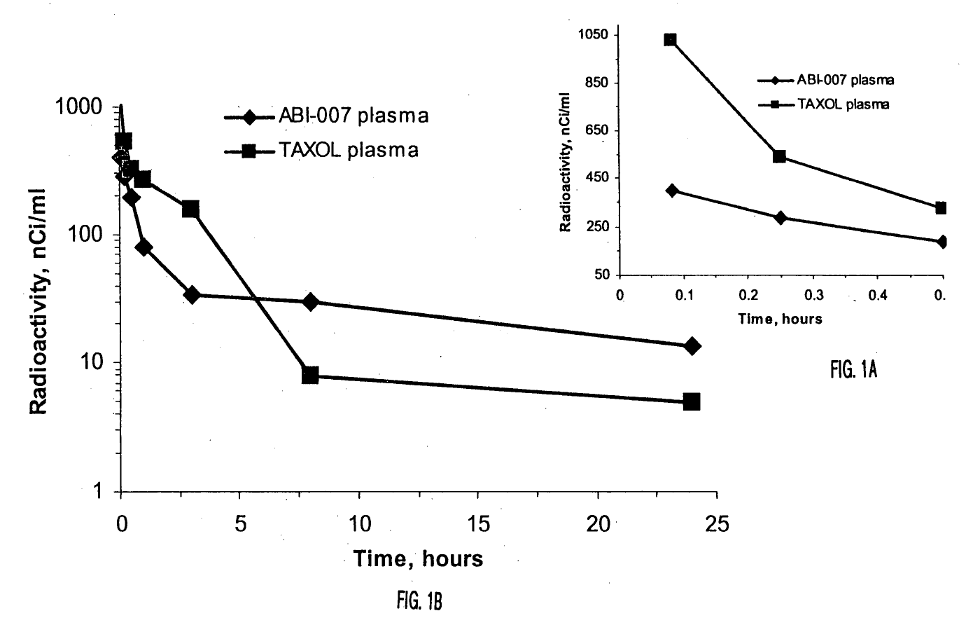

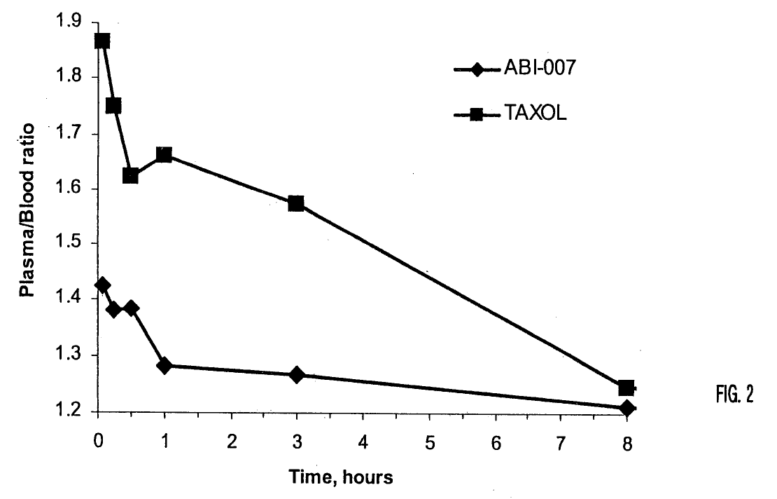

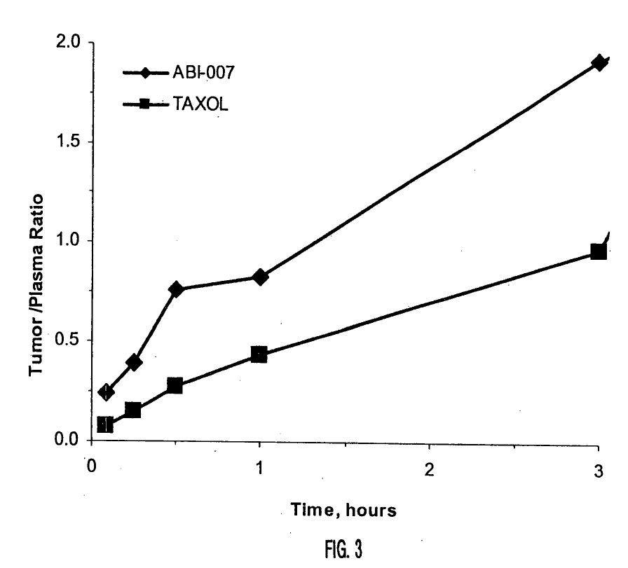

Radioactivity was determined as nCi/ml of whole blood and plasma, and nCi/g of tumor tissue. Results are presented in FIGS. 1, 2 and 3, and are standardized for radioactivity and paclitaxel dose. The data from these studies are also presented in the following tables.

PHARMACOKINETIC PARAMETERS FOR WHOLE-BLOOD, PLASMA AND
TUMOR DISTRIBUTION OF ³H-PACLITAXEL IN ABI-007 VS TAXOL

| | | | | | | | | | | New | |
|---|---|---|---|---|---|---|---|---|---|---|---|
| | AUC_{0-Inf} (nCi hr/mL or g) | | | | AUC₀₋₂₄ (nCi hr/mL or g) | | | | Cₘₐₓ (nCi/mL or g) | | |
| | Blood | Plasma | Tumor | | Blood | Plasma | Tumor | | Blood | Plasma | Tumor |
| ABI-007 | 939 | 1161 | 5869 | ABI-007 | 656 | 836 | 2156 | ABI-007 | 328 | 473 | 144 |
| Taxol | 871 | 1438 | 3716 | Taxol | 849 | 1415 | 1804 | Taxol | 752 | 1427 | 117 |
| Ratio | 1.08 | 0.81 | 1.58 | Ratio | 0.77 | 0.59 | 1.20 | Ratio | 0.44 | 0.33 | 1.23 |

| | |
|---|---|
| TAXOL: high Plasma AUC - paclitaxel is trapped in cremophor | ABI-007: Substantially lower Cmax in Plasma, blood implies |
| micelles | rapid distribution into cells and tissues |
| ABI-007: higher Tumor AUC (exposure), pac distributed into | ABI-007: higher Tumor Cmax - more effective tumor kill |
| cells/tissues | |

| | tₘₐₓ (hours) | | | | t½ (hours) | | | | Vdss (mL/kg) | | |
|---|---|---|---|---|---|---|---|---|---|---|---|
| | Blood | Plasma | Tumor | | Blood | Plasma | Tumor | | Blood | Plasma | Tumor |
| ABI-007 | 0 | 0 | 0.5 | ABI-007 | 17.1 | 16.1 | 40.2 | ABI-007 | 6939 | 5180 | NA |
| Taxol | 0 | 0 | 3 | Taxol | 4.0 | 3.3 | 24.1 | Taxol | 1409 | 692 | NA |

| | | | | | | |
|---|---|---|---|---|---|---|
| Ratio | 4.28 | 4.88 | 1.67 | Ratio | 4.92 | 7.49 |

| | | |
|---|---|---|
| ABI-007: Substantially lower | ABI-007: Prolonged half life | ABI-007: Substantially higher |
| tumor tmax indicates rapid | relative to Taxol in blood, plasma | volume of distribution indicating |
| uptake of paclitaxel into tumor | and tumor may result in higher | extrensive distribution into tissues |
| relative to taxol | antitumor activity | relative to Taxol |

Further studies demonstrate that after 24 hours, the active ingredient of the parent molecule, paclitaxel, remains present in the bloodstream, at double the concentration of Taxol. In studies comparing radiolabelled paclitaxel in Taxol vs ABI-007, direct measurements reveal increased and prolonged levels of paclitaxel in the tumors of animals receiving ABI-007.

### EXAMPLE 2

### Toxicity Studies

Toxicity was assessed for Taxol, cremophor and ABI-007. ABI-007 was found to be 50-fold less toxic than Taxol, and 30-fold less toxic than the cremophor vehicle alone, as illustrated in the following table:

| Agent | LD₅₀, mg/kg |
|---|---|
| Taxol | 9.4 |
| Cremophor | 13.7 |
| ABI-007 | 448.5 |

### EXAMPLE 3

### In Vivo Tumor Xenografts

Human tumor fragments were implanted subcutaneously in female athymic mice. Treatment was initiated when tumors reached about 150 mm³. The mice received either CONTROL (saline), ABI-007 (4 dose levels: 13.4, 20, 30 and 45 mg/kg) or TAXOL (3 dose levels: 13.4, 20, and 30 mg/kg) administered I.V. daily for 5 days. Saline was used as the diluent for both drugs.

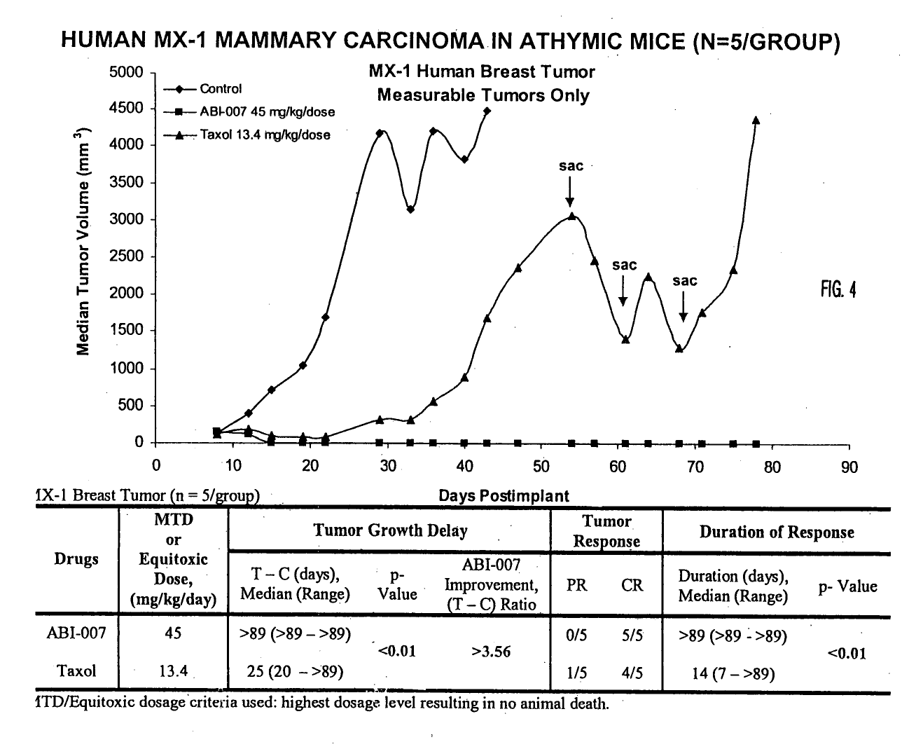

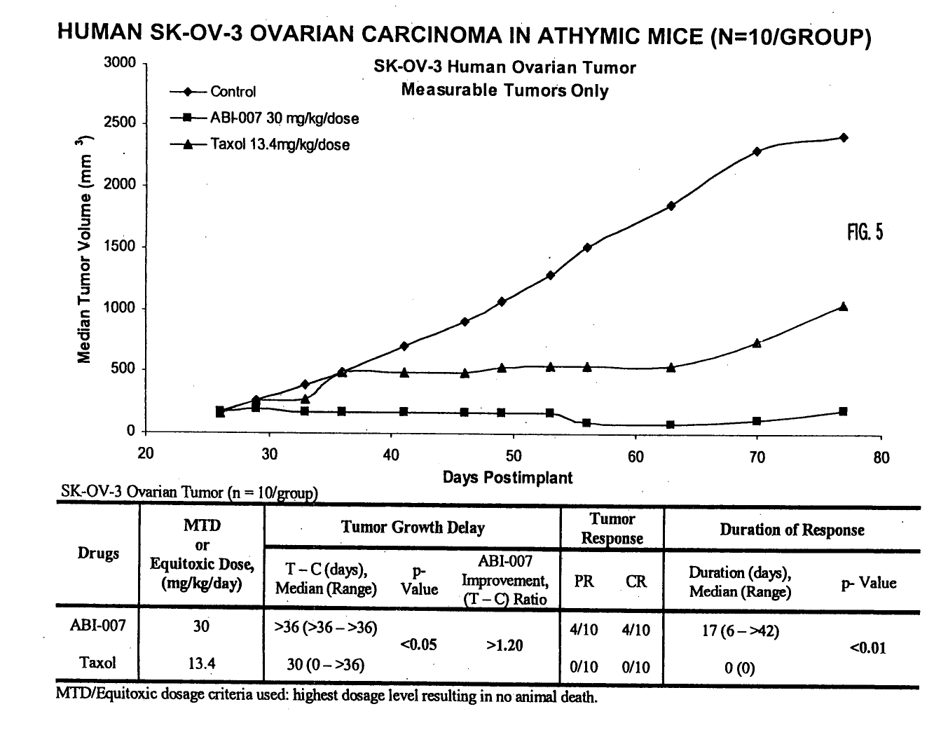

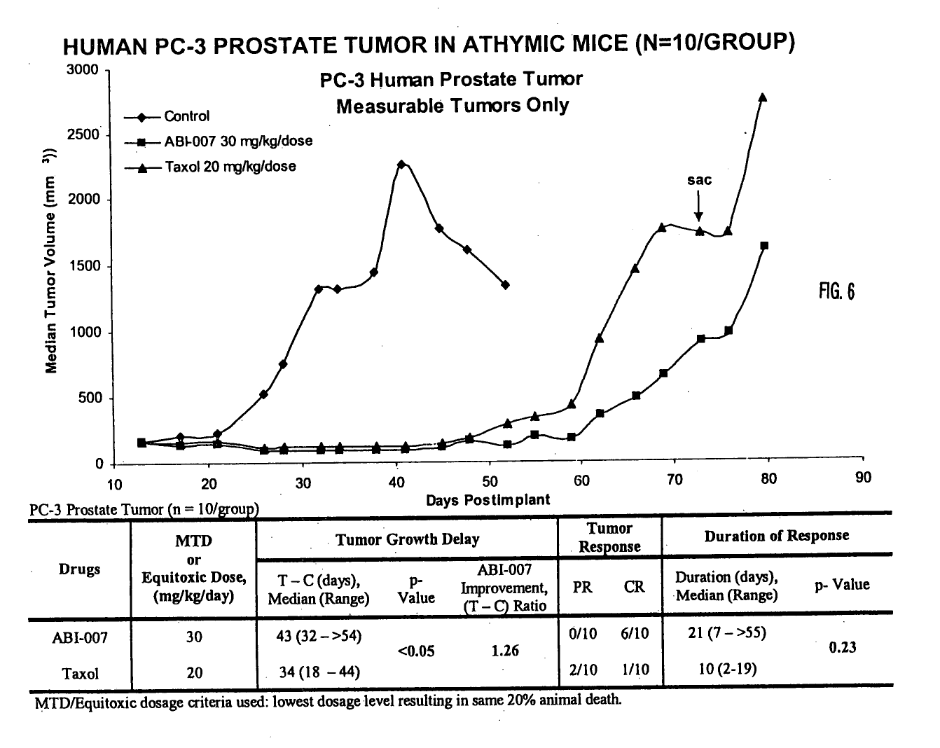

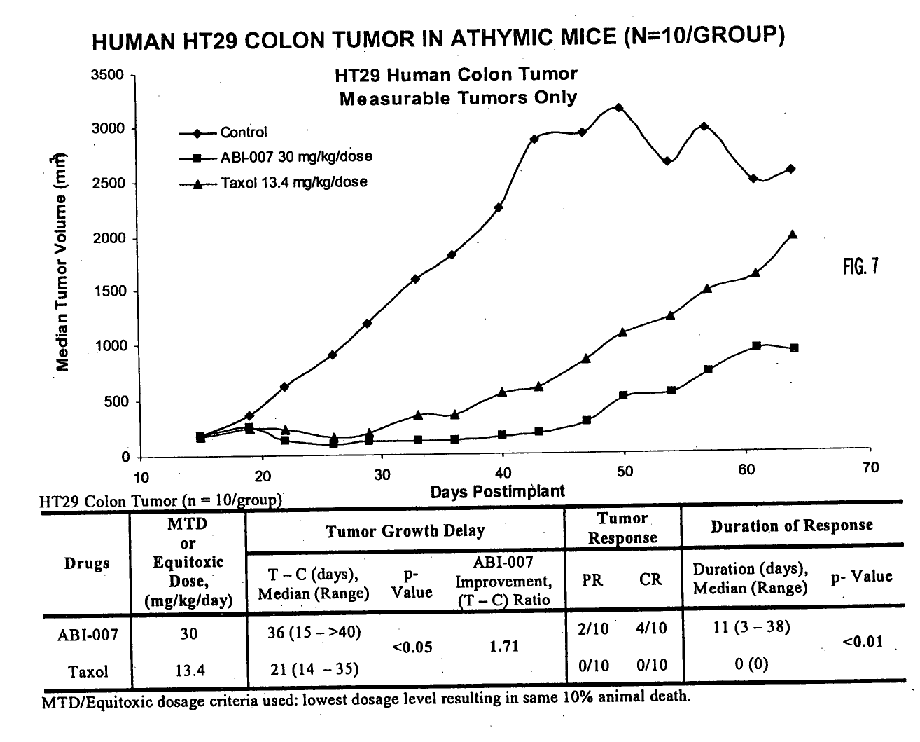

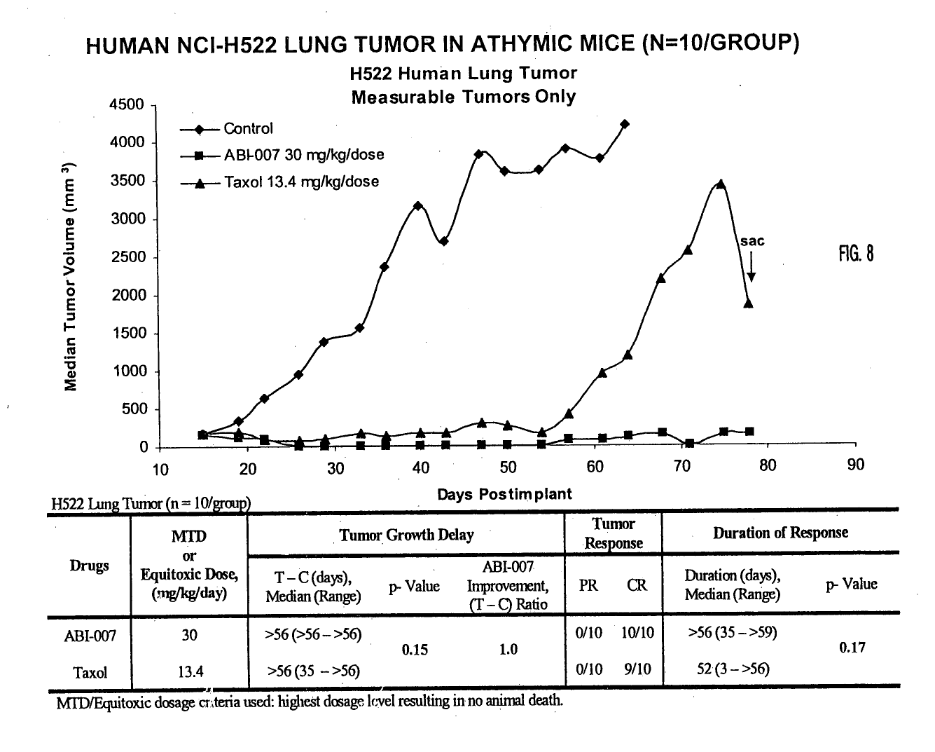

Determination of Equitoxic dose or MTD: The Equitoxic dose or MTD for each drug was determined by satisfying one of the following criteria:
a) Dose for each drug that resulted in similar body weight loss (≦20%) if no deaths were seen;
b) If body weight loss could not be matched, the highest dose at which no deaths were seen;
If neither a) nor b) could be satisfied, the lowest dose that resulted in similar death rate. Tumor response to the drugs was compared at the Equitoxic dose or MTD established as above. Results for several different tumor types are presented in FIGS. 4-8.

### EXAMPLE 4

### Clinical Studies

i. Entrappment of Paclitaxel By Cremophor

Working independently at Rotterdam Cancer Institute, Dr. Alex Sparreboom has reported in a series of pharmacokinetic studies involving patients receiving Taxol that cremophor “causes a profound alteration of paclitaxel accumulation in erythrocytes in a concentration-dependant manner by reducing the free drug fraction available for cellular partitioning.” He has further found that the drug trapping occurs in micelles and that these micelles act as the principal carrier of paclitaxel in the systemic circulation. Since that publication these findings have been independently confirmed by two other groups.

ii. Improved Clinical Activity With ABI-007

Data from Phase II shows both increased effiacacy in metastatic breast cancer patients. When compared to the published literature of response rates to Taxol, the study results showed a dramatic-difference in both response rates and time of response as well as evidence of reduced toxicities associated with ABI-007. Further details can be obtained by reviewing the posters presented at ASCO.

Although the present invention has been described in conjunction with the embodiments above, it is to be noted that various changes and modifications are apparent to those who are skilled in the art. Such changes and modifications are to be understood as included within the scope of the present invention defined by the appended claims.

1. A method for the delivery of a substantially water insoluble pharmacologically active agent to a subject in need thereof, said method comprising administering an effective amount of a formulation according to claim 12 to said subject.

2. A method according to claim 1 wherein said agent is paclitaxel.

3. A method according to claim 1 wherein said pharmaceutically acceptable carrier is albumin.

4. A method according to claim 1 wherein said formulation is administered by oral, intravenous, subcutaneous, intraperitoneal, intrathecal, intramuscular, intracranial, inhalational, topical, transdermal, rectal, or pessary route of administration.

5-7. (canceled)

8. A method to reduce entrapment of a substantially water insoluble pharmacologically active agent in vehicle employed for delivery thereof, said method comprising employing a formulation according to claim 12 for delivery of said agent, thereby reducing entrapment of said substantially water insoluble pharmacologically active agent relative to a formulation comprising the same agent and a carrier containing micelle-forming components.

9. A method to prolong exposure of a subject to a substantially water insoluble pharmacologically active agent upon administration thereof to a subject in need thereof, said method comprising combining said agent with a pharmaceutically acceptable carrier which is free of micelle-forming components to produce a formulation according to claim 12 prior to delivery thereof, thereby prolonging exposure of said subject to said substantially water insoluble pharmacologically active agent relative to a formulation comprising the same agent and a carrier containing micelle-forming components.

10. A method to facilitate transport of a substantially water insoluble pharmacologically active agent across cell membranes and/or into the cellular compartment upon administration thereof to a subject in need thereof, said method comprising combining said agent with a pharmaceutically acceptable carrier which is free of micelle-forming components to produce a formulation according to claim 12 prior to delivery thereof, thereby facilitating transport of said substantially water insoluble pharmacologically active agent across cell membranes relative to transport of a formulation comprising the same agent and a carrier containing micelle-forming components.

11. (canceled)

12. A formulation comprising a substantially water insoluble pharmacologically active agent and a pharmaceutically acceptable carrier which is free of micelle-forming components, wherein said formulation is characterized by one or more of the following:
wherein said formulation provides a higher concentration of said agent in the cellular compartment than a formulation of the same agent with a micelle-forming component;
wherein said formulation provides increased intra-cellular availability of said agent relative to a formulation of the same agent with a micelle-forming component;
wherein said formulation provides prolonged activity of said agent relative to a formulation of the same agent with a micelle-forming component;
wherein said formulation facilitates delivery of said agent to red blood cells, relative to delivery of said agent to red blood cells by a formulation comprising the same agent and a carrier containing micelle-forming components;
wherein said formulation releases a portion of said agent contained therein to the lipid membrane of a cell:
wherein said formulation provides reduced levels of said agent in the bloodstream relative to a formulation of the same agent with a micelle-forming component;
wherein said formulation delivers said agent to the bloodstream over an extended period of time relative to a formulation of the same agent with a micelle-forming component;
wherein the rate of metabolism of said agent in said formulation is reduced relative to the rate of metabolism of said agent in a formulation with a micelle-forming component;
wherein said agent has a longer half life in said formulation relative to the half life of said agent in a formulation with a micelle-forming component;
wherein said formulation provides a higher red blood cell/plasma ratio of said agent than does a formulation of the same agent with a micelle-forming component;
wherein said formulation provides a higher tumor/plasma ratio of said agent than does a formulation of the same agent with a micelle-forming component;
wherein the area under the curve for delivery of said agent to a tumor via said formulation is higher than the area under the curve for delivery of said agent to a tumor via a formulation of the same agent with a micelle-forming component;
wherein said formulation provides a higher concentration maximum (Cₘₐₓ) for said agent in tumor cells than does a formulation of the same agent with a micelle-forming component;
wherein said formulation provides a lower concentration maximum (Cₘₐₓ) for said agent in plasma than does a formulation of the same agent with a micelle-forming component;
wherein said formulation provides more rapid uptake of said agent by tumor cells than does a formulation of the same agent with a micelle-forming component, and
wherein said formulation enhances delivery of said agent to tissue, relative to a formulation of the same agent with a micelle-forming component.

13. A formulation according to claim 12, wherein said formulation provides a higher concentration of said agent in the cellular compartment than a formulation of the same agent with a micelle-forming component and/or said formulation provides increased intra-cellular availability of said agent relative to a formulation of the same agent with a micelle-forming component.

14. A formulation according to claim 12, wherein said formulation provides prolonged activity of said agent relative to a formulation of the same agent with a micelle-forming component and/or said formulation delivers said agent to the bloodstream over an extended period of time relative to a formulation of the same agent with a micelle-forming component.

15. A formulation according to claim 12, wherein said formulation facilitates delivery of said agent to red blood cells, relative to delivery of said agent to red blood cells by a formulation comprising the same agent and a carrier containing micelle-forming components and/or said formulation provides reduced levels of said agent in the bloodstream relative to a formulation of the same agent with a micelle-forming component.

16. A formulation according to claim 12, wherein said formulation releases a portion of said agent contained therein to the lipid membrane of a cell.

17-18. (canceled)

19. A formulation according to claim 12, wherein the rate of metabolism of said agent in said formulation is reduced relative to the rate of metabolism of said agent in a formulation with a micelle-forming component and/or said agent has a longer half life in said formulation relative to the half life of said agent in a formulation with a micelle-forming, component.

20. (canceled)

21. A formulation according to claim 12, wherein said formulation provides a higher red blood cell/plasma ratio of said agent than does a formulation of the same agent with a micelle-forming component and/or said formulation provides a higher tumor/plasma ratio of said agent than does a formulation of the same agent with a micelle-forming component.

22. (canceled)

23. A formulation according to claim 12, wherein the area under the curve for delivery of said agent to a tumor via said formulation is higher than the area under the curve for delivery of said agent to a tumor via a formulation of the same agent with a micelle-forming component.

24. A formulation according to claim 12, wherein said formulation provides a higher concentration maximum (Cₘₐₓ) for said agent in tumor cells than does a formulation of the same agent with a micelle-forming component and/or said formulation provides a lower concentration maximum (Cₘₐₓ) for said agent in plasma than does a formulation of the same agent with a micelle-forming component.

25. (canceled)

26. A formulation according to claim 12, wherein said formulation provides more rapid uptake of said agent by tumor cells than does a formulation of the same agent with a micelle-forming component.

27. A formulation according to claim 12, wherein said formulation enhances delivery of said agent to tissue, relative to a formulation of the same agent with a micelle-forming component.

28. A formulation according to claim 27 wherein said tissue is a tumor.

29. A formulation according to claim 27 wherein said tissue is peritoneal tissue, bladder tissue or lung tissue.
